(19) United States
(12) Patent Application Publication
(10) Pub. No.: US 2004/0076595 A1
(43) Pub. Date: Apr. 22, 2004

(54) Hair conditioning composition comprising thickening polymer and cationic surfactant

(75) Inventor: Golam Faruque Khan, Kobe (JP)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/683,830

(22) Filed: Oct. 10, 2003

Related U.S. Application Data
(60) Provisional application No. 60/417,710, filed on Oct. 10, 2002.
(60) Provisional application No. 60/476,706, filed on Jun. 6, 2003.

(51) Int. Cl. A61K 7/06; A61K 7/11; A61K 7/075; A61K 7/08

(52) U.S. Cl. 424/70.11; 424/70.28

(57) ABSTRACT

Disclosed is a hair conditioning composition comprising: (a) a thickening polymer system selected from the group consisting of a cationic thickening polymer, a nonionic thickening polymer, and mixtures thereof; (b) cationic surfactant system selected from the group consisting of one cationic surfactant, a mixture of two or more cationic surfactants, and a mixture of a cationic surfactant and a nonionic surfactant; and (c) an aqueous carrier; wherein the composition is substantially free of a water-insoluble high melting point oily compound and an anionic compound. Preferably, the conditioning composition further contains a silicone compound selected from the group consisting of a water-soluble silicone compound, a silicone nanoemulsion with an average particle diameter of less than 220 nm, and mixtures thereof. The conditioning composition of the present invention has a suitable rheology for conditioning compositions and provides conditioning benefits. The conditioning composition of the present invention is especially suitable for rinse-off use.

### CROSS REFERENCE TO RELATED APPLICATION

This application claims the benefit of U.S. Provisional application No. 60/417710, filed on Oct. 10, 2002 and U.S. Provisional application No. 60/476706, filed on Jun. 6, 2003.

### FIELD OF THE INVENTION

The present invention relates to hair conditioning compositions comprising a thickening polymer system, a cationic surfactant system, and the composition being substantially free of water-insoluble high melting point oily compounds and anionic compounds. The conditioning composition of the present invention has a suitable rheology for conditioning compositions and provides conditioning benefits. The conditioning composition of the present invention is especially suitable for rinse-off use.

### BACKGROUND OF THE INVENTION

A variety of approaches have been developed to condition the hair. A common method of providing conditioning benefit is through the use of conditioning agents such as cationic surfactants and polymers, high melting point fatty compounds, low melting point oils, silicone compounds, and mixtures thereof. Most of these conditioning agents are known to provide various conditioning benefits. For example, some cationic surfactants, when used together with some high melting point fatty compounds, are believed to provide a gel matrix which has a suitable rheology for conditioning compositions and which is suitable for providing a variety of conditioning benefits, especially when used for hair care products, such as slippery feel, softness and reduced tangling on wet hair and softness and moisturized feel on the hair when they are dried.

There exists a need for achieving the suitable rheology for conditioning compositions by other methods than forming the above gel matrix, while maintaining the conditioning benefits of the gel matrix.

Additionally, most of the above conditioning agents are also known to make the composition opaque. Thus, there is a need for conditioning compositions having a clear product appearance i.e., transparent or translucent product appearance.

Furthermore, most of the above conditioning agents are also known to weigh down the hair. For consumers who desire maintaining or increasing hair volume such as consumers having fine hair, the hair weighing down is not desirable. Thus, there is a need for hair conditioning compositions which do not weigh down the hair while providing conditioning benefits.

There also exists a need for conditioning compositions which consumers feel are easy to rinse-off while providing conditioning benefits, when the compositions are used in a form of rinse-off products. Conditioner compositions containing the above gel matrix also provide long-lasting slippery feel when rinsing the hair. Thus, there is a need for conditioning compositions which can easily leave the hair with a clean feel when rinsing the hair, while depositing sufficient amount of conditioning agents on the hair.

Based on the foregoing, there remains a need for conditioning compositions which have a suitable rheology for conditioning compositions by other methods than a gel matrix comprised by cationic surfactants and high melting point fatty compounds, while providing conditioning benefits, especially softness and reduced tangling on wet hair. There is also a need for such conditioning compositions which are suitable for providing further benefits such as, clear product appearance, not weighing down the hair, and easy to rinse-off feel, while providing the above rheological and conditioning benefits.

None of the existing art provides all of the advantages and benefits of the present invention.

### SUMMARY OF THE INVENTION

The present invention is directed to a hair conditioning composition comprising by weight:

(a) from about 0.01% to about 10% of a thickening polymer system selected from the group consisting of a cationic thickening polymer, a nonionic thickening polymer, and mixtures thereof;

(b) from about 0.05% to about 10% of a cationic surfactant system selected from the group consisting of one cationic surfactant, a mixture of two or more cationic surfactants, and a mixture of a cationic surfactant and a nonionic surfactant; and

(c) an aqueous carrier;

wherein the composition is substantially free of a water-insoluble high melting point oily compound and an anionic compound.

The present invention is also directed to a hair conditioning composition comprising by weight:

(a) from about 0.5% to about 5% of a thickening polymer system selected from the group consisting of a cationic thickening polymer, a nonionic thickening polymer, and mixtures thereof;

(b) from about 0.25% to about 7% of a cationic surfactant system selected from the group consisting of one cationic surfactant, a mixture of two or more cationic surfactants, and a mixture of a cationic surfactant and a nonionic surfactant;

(c) from about 0.1% to about 10% of a silicone compound selected from the group consisting of a water-soluble silicone compound, a silicone nanoemulsion with an average particle diameter of less than 220 nm, and mixtures thereof; and

(d) an aqueous carrier;

wherein the composition is substantially free of a water-insoluble high melting point oily compound and an anionic compound.

The present invention is further directed to a hair conditioning composition comprising by weight:

(a) from about 0.5% to about 5% of a nonionic thickening polymer;

(b) from about 0.25% to about 7% of a cationic surfactant system selected from the group consisting of one cationic surfactant, a mixture of two or more cationic surfactant, and mixture of a cationic surfactant and a nonionic surfactant;

(c) from about 0.1% to about 10% of a silicone compound selected from the group consisting of a water-soluble silicone compound, a silicone nanoemulsion with an average particle diameter of less than 220 nm, and mixtures thereof; and

(d) an aqueous carrier;

wherein the composition is substantially free of a water-insoluble high melting point oily compound, an anionic compound, and a cationic thickening polymer.

These and other features, aspects, and advantages of the present invention will become better understood from a reading of the following description, and appended claims.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

Compositions

The present invention is directed to a hair conditioning composition comprises by weight:

The present invention is directed to a hair conditioning composition (Composition A) comprising by weight:

(a) from about 0.01% to about 10% of a thickening polymer system selected from the group consisting of a cationic thickening polymer, a nonionic thickening polymer, and mixtures thereof;

(b) from about 0.05% to about 10% of a cationic surfactant system selected from the group consisting of one cationic surfactant, a mixture of two or more cationic surfactants, and a mixture of a cationic surfactant and a nonionic surfactant; and

(c) an aqueous carrier;

wherein the composition is substantially free of a water-insoluble high melting point oily compound and an anionic compound.

The present invention is also directed to a hair conditioning composition (Composition B) comprising by weight:

(a) from about 0.5% to about 5% of a thickening polymer system selected from the group consisting of a cationic thickening polymer, a nonionic thickening polymer, and mixtures thereof;

(b) from about 0.25% to about 7% of a cationic surfactant system selected from the group consisting of one cationic surfactant, a mixture of two or more cationic surfactants, and a mixture of a cationic surfactant and a non ionic surfactant;

(c) from about 0.1% to about 10% of a silicone compound selected from the group consisting of a water-soluble silicone compound, a silicone nanoemulsion with an average particle diameter of less than 220 nm, and mixtures thereof; and

(d) an aqueous carrier;

wherein the composition is substantially free of a water-insoluble high melting point oily compound and an anionic compound.

The present invention is further directed to a hair conditioning composition (Composition C) comprising by weight:

(a) from about 0.5% to about 5% of a nonionic thickening polymer;

(b) from about 0.25% to about 7% of a cationic surfactant system selected from the group consisting of one cationic surfactant, a mixture of two or more cationic surfactant, and mixture of a cationic surfactant and a nonionic surfactant;

(c) from about 0.1% to about 10% of a silicone compound selected from the group consisting of a water-soluble silicone compound, a silicone nanoemulsion with an average particle diameter of less than 220 nm, and mixtures thereof; and

(d) an aqueous carrier;

wherein the composition is substantially free of a water-insoluble high melting point oily compound, an anionic compound, and a cationic thickening polymer.

The conditioning compositions of the present invention have a suitable rheology for conditioning compositions and provide conditioning benefits, especially softness and reduced tangling on wet hair. It is believed that; by the combination of the cationic and nonionic thickening polymers and the cationic surfactant, the composition of the present invention can provide a suitable rheology for conditioning compositions without the existence of a gel matrix comprised by cationic surfactants and high melting point fatty compounds, while providing conditioning benefits, especially softness and reduced tangling on wet hair.

The conditioning compositions of the present invention are suitable for providing further benefits such as clear product appearance, not weighing down the hair, and easy to rinse-off feel, while providing the above rheological and conditioning benefits. Thus, the composition of the present invention can provide clear product appearance in addition to the above rheological and conditioning benefits. The composition of the present invention can provide the above rheological and conditioning benefits while not weighing down the hair. Furthermore, when used in a form of rinse-off products, the composition of the present invention can provide easy to rinse-off feel while providing the above rheological and conditioning benefits.

In the present invention, the composition being “substantially free of water-insoluble high melting point oily compound” means that the composition includes 1.0% or less, preferably 0.5% or less, more preferably 0.1% or less, still more preferably 0% of water-insoluble high melting point oily compounds. The water-insoluble high melting point oily compounds herein are those having a melting point of at least about 25° C., and a solubility in water at 25° C. of less than about 1 g/100 g water, preferably less than about 0.5 g/100 g water, more preferably less than about 0 g/100 g water. Such water-insoluble high melting point oily compound include, for example, fatty alcohols such as cetyl alcohol and stearyl alcohol, fatty acids such as stearic acid, fatty alcohol derivatives and fatty acid derivatives such as cetyl palmitate, hydrocarbons such as waxes, steroids such as cholesterol, and mixtures thereof.

In the present invention, the composition being “substantially free of anionic compounds” means that the composition includes 1% or less, preferably 0.5% or less, more preferably 0% of anionic compounds. Anionic compounds herein include anionic surfactants and anionic polymers.

In the present invention, Composition C is substantially free of cationic thickening polymers. The composition being “substantially free of cationic thickening polymers” means that the composition includes 1.0% or less, preferably 0.5% or less, more preferably 0.1% or less, still more preferably 0% of cationic thickening polymers.

In the present invention, the composition is preferably transparent or translucent, and more preferably transparent. In the present invention, the composition being “transparent” means that the composition has a transmittance of more than about 30%, preferably more than about 50%, more preferably more than about 80%. The transmittance can be measured by any standard spectrophotometers comparing with deionized water. In the present invention, the composition being “translucent” means that the composition has both (i) a turbidity of less than about 3,000 NTU (Nephelometric Turbidity Units) and (ii) a transmittance of less than 50%, preferably less than about 30%. The NTU values are measured using the Hach 2100N Laboratory Turbidimeter calibrated with Formazin standards, available from Hach Company.

Preferably Composition A further comprises a silicone compound, wherein the silicone compound is preferably selected from the group consisting of a water-soluble silicone compound, a silicone nanoemulsion having a particle size of less than 220 nm, and mixtures thereof.

The compositions may further contain a humectant such as polyethylene glycol. The compositions may further contain an antifoaming agent.

Thickening Polymer

The compositions of the present invention comprise a thickening polymer. The thickening polymers useful herein are those which can provide appropriate viscosity and rheology properties to the composition, so that the composition of the present composition has a suitable viscosity of from about 1, 000 to about 100,000 mPa·s (cps), preferably from about 1,000 to about 50,000 mPa·s (cps), more preferably from about 2,000 to about 50,000 mPa·s (cps), still more preferably from about 5,000 to about 20,000 mPa·s (cps). The viscosity herein can be suitably measured by Brookfield RVT at a shear rate of 2·s-1 at 26.7° C.

The composition of the present invention comprises by weight of from about 0.01% to about 10%, preferably from about 0.05% to about 8%, more preferably from about 0.1% to about 5%, still more preferably from about 0.5% to about 4%, even more preferably from about 0.6% to about 3%, highly preferably from about 1.0% to about 2.5%, of total thickening polymers.

A variety of thickening polymers can be used in the compositions of the present invention. Thickening polymers useful herein include, for example, cellulose and its derivatives such as cellulose ethers, hydrophobically modified cellulose ethers, and quaternized celluloses; guar gums including cationic guar gums and nonionic guar gums; crosslinked polymers such as nonionic crosslinked polymers and cationic crosslinked polymers; and acrylate polymers such as sodium polyacrylate, polyethylacrylate, and polyacrylamide. The thickening polymers useful herein may include the polymers disclosed below under the title “CATIONIC CONDITIONING POLYMER”. Among a variety of thickening polymers, the composition of the present invention comprises a cationic thickening polymer, a nonionic thickening polymer, or mixtures thereof.

In one preferred embodiment, the composition of the present invention comprises a thickening polymer system comprising a mixture of a cationic thickening polymer and a nonionic thickening polymer, in view of providing conditioning benefits. Such thickening system is a cationic system. In the present invention, what is meant by “a cationic system” is that the system comprises at least one cationic thickening agent. In such preferable cationic thickening systems, the composition is substantially free of anionic compounds such as anionic surfactants and anionic polymers. In the present invention, the composition being “substantially free of anionic compounds” means that the composition includes 1% or less, preferably 0.5% or less, more preferably 0% of anionic compounds.

In another preferred embodiment, the composition of the present invention comprises a nonionic thickening polymer, and the composition is substantially free of cationic thickening polymer.

The thickening polymer or system useful herein has improved compatibility with cationic conditioning agents such as cationic surfactants.

Guar Polymer

In the composition, guar polymers are preferably used among a variety of thickening polymers.

The cationic guar polymer useful herein has a level of cationic substitution of preferably from about 0.05 to about 0.3, and a molecular weight of preferably from about 100,000 to about 3,000,000, more preferably from about 1,000,000 to about 2,400,000. In the present invention, the cationic substitution level is an integer obtained by following equation: the number of cationically substituted hydroxyl groups divided by the number of all hydroxyl groups before substitution. Commercially available cationic guar polymers useful herein include, for example, guar hydroxypropyl trimonium chloride (having a molecular weight of about 1,500,000, and a level of cationic substitution of about 0.15) having a tradename Jaguar Excel available from Rhodia, guar hydroxypropyl trimonium chloride (having a molecular weight of from about 1,000,000 to about 1,500,000, and a level of cationic substitution of about 0.25) having a tradename Jaguar CHJ available from Rhodia. The cationic guar polymers can be included in the composition at a level by weight of preferably from about 0.1% to 4%, more preferably from 0.3% to 2%.

The nonionic guar polymer useful herein has a molecular weight of preferably from about 500,000 to about 4,000,000, more preferably from about 1,000,000 to about 4,000,000, still more preferably from about 1,600,000 to about 3,000,000, even more preferably from about 1,900,000 to about 2,800,000. Commercially available nonionic guar polymers useful herein include, for example, that having a molecular weight of about 2,000,000 and having a tradename Jaguar HP-105 available from Rhodia. The nonionic guar polymer can be included in the composition at a level by weight of preferably from about 0.1% to 3%, more preferably from about 0.3% to 2.5%.

Cationic Surfactant System

The compositions of the present invention comprise a cationic surfactant system. The cationic surfactant system is included in the composition at a level by weight of from about 0.05% to about 10%, preferably from about 0.25% to about 7%, more preferably from about 0.3% to about 5%, still more preferably from about 0.4% to about 3%.

The cationic surfactant system is selected from the group consisting of one cationic surfactant, a mixture of two or more cationic surfactants, a mixture of a cationic surfactant and a nonionic surfactant. When the composition comprises a thickening polymer system comprising a cationic thickening polymer and a nonionic thickening polymer, the cationic surfactant system is preferably one cationic surfactant or a mixture of two or more cationic surfactants. In Composition C, the cationic surfactant system is preferably a mixture of two or more cationic surfactants, or a mixture of a cationic surfactant and a non ionic surfactant.

Cationic Surfactant

In the composition of the present invention, mono-alkyl trimethyl ammonium salts are preferably used among a variety of cationic surfactants described below. The mono-alkyl trimethyl ammonium salts useful herein are those in which the alkyl has from 12 to 28 carbon atoms, preferably from 16 to 22 carbon atoms. The mono-alkyl trimethyl ammonium salts useful herein includes, for example, cetyl trimethyl ammonium chloride, stearyl trimethyl ammonium chloride.

Cationic surfactants useful herein include, for example, those corresponding to the general formula (I):

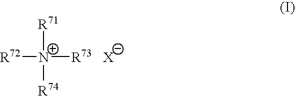

wherein at least one of R⁷¹, R⁷², R⁷³ and R⁷⁴ is selected from an aliphatic group of from 8 to 30 carbon atoms or an aromatic, alkoxy, polyoxyalkylene, alkylamido, hydroxyalkyl, aryl or alkylaryl group having up to about 22 carbon atoms, the remainder of R⁷¹, R⁷², R⁷³ and R⁷⁴ are independently selected from an aliphatic group of from 1 to about 22 carbon atoms or an aromatic, alkoxy, polyoxyalkylene, alkylamido, hydroxyalkyl, aryl or alkylaryl group having up to about 22 carbon atoms; and X is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The aliphatic groups can contain, in addition to carbon and hydrogen atoms, ether linkages, and other groups such as amino groups. The longer chain aliphatic groups, e.g., those of about 12 carbons, or higher, can be saturated or unsaturated. Preferred is when R⁷¹, R⁷², R⁷³ and R⁷⁴ are independently selected from C₁ to about C₂₂ alkyl. Nonlimiting examples of cationic surfactants useful in the present invention include the materials having the following CTFA designations: quaternium-8, quaternium-14, quaternium-18, quaternium-18 methosulfate, quaternium-24, and mixtures thereof.

Among the cationic surfactants of general formula (I), preferred are those containing in the molecule at least one alkyl chain having at least 16 carbons. Nonlimiting examples of such preferred cationic surfactants include: behenyl trimethyl ammonium chloride available, for example, with tradename Genamine KDMP from Clariant, with tradename INCROQUAT TMC-80 from Croda, and with tradename ECONOL TM22 from Sanyo Kasei; cetyl trimethyl ammonium chloride available, for example, with tradename CTAC 30KC from KCI, and with tradename CA-2350 from Nikko Chemicals; stearyl trimethyl ammonium chloride available, for example, with tradename Genamine STACP from Clariant; olealkonium chloride available, for example, with tradename Incroquat O-50 from Croda; hydrogenated tallow alkyl trimethyl ammonium chloride, dialkyl (14-18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, di(behenyl/arachidyl) dimethyl ammonium chloride, dibehenyl dimethyl ammonium chloride, stearyl dimethyl benzyl ammonium chloride, stearyl propyleneglycol phosphate dimethyl ammonium chloride, stearoyl amidopropyl dimethyl benzyl ammonium chloride, stearoyl amidopropyl dimethyl (myristylacetate) ammonium chloride, and N-(stearoyl colamino formyl methyl) pyridinium chloride.

Also preferred are hydrophilically substituted cationic surfactants in which at least one of the substituents contain one or more aromatic, ether, ester, amido, or amino moieties present as substituents or as linkages in the radical chain, wherein at least one of the R⁷¹-R⁷⁴ radicals contain one or more hydrophilic moieties selected from alkoxy (preferably C₁-C₃ alkoxy), polyoxyalkylene (preferably C₁-C₃ polyoxyalkylene), alkylamido, hydroxyalkyl, alkylester, and combinations thereof. Preferably, the hydrophilically substituted cationic conditioning surfactant contains from 2 to about 10 nonionic hydrophile moieties located within the above stated ranges. Highly preferred hydrophilically substituted cationic surfactants include dialkylamido ethyl hydroxyethylmonium salt, dialkylamidoethyl dimonium salt, dialkyloyl ethyl hydroxyethylmonium salt, dialkyloyl ethyldimonium salt, and mixtures thereof; for example, commercially available under the following tradenames; VARISOFT 110, VARISOFT 222, VARIQUAT K1215 and VARIQUAT 638 from Witco Chemical, MACKPRO KLP, MACKPRO WLW, MACKPRO MLP, MACKPRO NSP, MACKPRO NLW, MACKPRO WWP, MACKPRO NLP, MACKPRO SLP from McIntyre, ETHOQUAD 18/25, ETHOQUAD O/12PG, ETHOQUAD C/25, ETHOQUAD S/25, and ETHODUOQUAD from Akzo, DEHYQUAT SP from Henkel, and ATLAS G265 from ICI Americas. Babassuamidopropalkonium Chloride available from Croda under the tradename Incroquat BA-85 is also preferably used in the composition.

Amines are suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Also useful are dimethylstearamine, dimethylsoyamine, soyamine, myristylamine, tridecylamine, ethylstearylamine, N-tallowpropane diamine, ethoxylated (with 5 moles of ethylene oxide) stearylamine, dihydroxyethylstearylamine, and arachidylbehenylamine. Useful amines in the present invention are disclosed in U.S. Pat. No. 4,275,055, Nachtigal, et al. These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; more preferably l-glutamic acid, lactic acid, citric acid. The amines herein are preferably partially neutralized with any of the acids at a molar ratio of the amine to the acid of from about 1:0.3 to about 1:2, more preferably from about 1:0.4 to about 1:1.

Nonionic Surfactant

Nonionic surfactants useful herein are those having a HLB value of from about 8 to about 20, preferably from about 10 to about 18, more preferably from about 12 to about 15. Among a variety of nonionic surfactants, highly preferred are ethylene glycol ethers of fatty alcohols. Among them, especially preferred are Ceteth-10, pareth-12, and Laureth-9.

Aqueous Carrier

The compositions of the present invention comprise an aqueous carrier. The level and species of the carrier are selected according to the compatibility with other components, and other desired characteristic of the product.

Preferably, the aqueous carrier is substantially water. Deionized water is preferably used. Water from natural sources including mineral cations can also be used, depending on the desired characteristic of the product. Generally, the compositions of the present invention comprise from about 20% to about 99%, preferably from about 40% to about 98%, and more preferably from about 50% to about 98% water.

The pH of the present composition is preferably from about 3 to about 9, more preferably from about 3 to about 7. Buffers and other pH adjusting agents can be included to achieve the desirable pH.

Silicone Compound

Preferably, the compositions of the present invention contain a silicone compound. The silicone compounds herein are preferably used at levels by weight of the compositions of from about 0.1% to about 20%, more preferably from about 0.15% to about 10%, still more preferably from about 0.2% to about 5%.

The silicone compounds for use herein will preferably have a viscosity of from about 100 to about 2,000,000 centistokes at 25° C. The viscosity can be measured by means of a glass capillary viscometer as set forth in Dow Corning Corporate Test Method CTM0004, Jul. 20, 1970.

Among a variety of silicone compounds, preferred are those selected from the group consisting of a water-soluble silicone compound, a silicone nanoemulsion, and mixtures thereof, in the composition of the present invention. In the present invention, a “water-soluble” silicone compound means that the silicone compound has a solubility in water at 25° C. of at least 0.05 g/100 g water, preferably at least 0.1 g/100 g water, more preferably at least 0.2 g/100 g water. In the present invention, a “water-soluble” silicone compound also means that the silicone compound is solublized in water in the presence of surfactants. Dimethicone copolyols, amodimethicone copolyols, and quaternized silicones can be water-soluble depending on the level of alkoxylate chains and quaternized groups in their structure. Commercially available water-soluble silicone compounds useful herein include, for example, dimethicone copolyol with a tradename Silicone DC-5330, and amidomethicone copolyol having a INCI name PEG-12 Methyl Ether/Lauroxy PEG-5 Amidopropyl Dimethicone available with tradenames Silicone BY16-906, and amodimethicone having an INCI name Bis (C13-15 Alkoxy) PG Amodimethicone available with a tradename DC2-8500, all from Dow Corning. In the present invention, a “nanoemulsion” of silicone compound means that the silicone compound is dispersed in the composition in the form of an emulsion having an average particle diameter of 220 nm or less, preferably 100 nm or less, more preferably 50 nm or less. Commercially available silicone compounds in the form of a nanoemulsion useful herein includes, for example, that with a tradename Silicone DC-8177 available from Dow Corning; quaternized silicone nanoemulsion with a tradename DC5-7133 available from Dow Corning; amodimethicone nanoemulsion with a tradename XS65-B6413 available from General Electric; and amodimethicone nanoemulsion with tradenames DC2-8168 and DC2-8194 available from Dow Corning.

Highly preferred water-soluble silicone compound useful herein is a hydrophobically modified amidomethicone copolyol having the following formula:

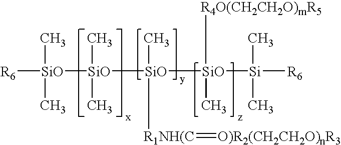

wherein R₁, R₂, R₄ are respectively C1-C3 alkyl, preferably ethyl; R₃ is an alkyl group having 8-22 carbon atoms, preferably 10-20 carbon atoms, more preferably 12-16 carbon atoms, even more preferably 12 carbon atoms; R₅ is H or C1-C3 alkyl, preferably methyl; R₆ is OH or CH₃, preferably methyl; n is an integer of 1-10, highly preferably 5; m is an integer of 2-20, highly preferably 12; n+m =3-30, preferably 5-25, more preferably 8-20, even more preferably 17; x is an integer from 200 to 500, preferably from 300 to 400; y is an integer from 5 to 40, preferably from 10 to 30; and z is 0 or an integer from 1 to 30, preferably from 5 to 20. Commercially available hydrophobically modified amidomethicone copolyols useful herein include, for example, that available from Dow Coming with a tradename BY16-906.

Other silicones hair conditioning properties may also be used in the composition. Such silicones include, for example, polyalkyl siloxanes such as polydimethylsiloxane from General Electric Company in their TSF 451 series and from Dow Corning in their Dow Corning SH200 series; polyaryl siloxanes; polyalkylaryl siloxanes; polyether siloxane copolymers; amino substituted silicones such as amodimethicone with tradename BY16-872 available from Dow Corning; quaternized silicones such as that available from Union Carbide under the tradename UCAR SILICONE ALE 56 and that available from Noveon with a tradename Ultrasil Q-Plus; and mixtures thereof.

Cationic Conditioning Polymer

The hair conditioning compositions of the present invention can further include cationic conditioning polymers. The cationic polymers hereof will generally have a weight average molecular weight which is at least about 5,000, typically at least about 10,000, and is less than about 10 million, preferably, the molecular weight is from about 100,000 to about 2 million. The cationic polymers useful herein may include the polymers disclosed above under the title “THICKENING POLYMER”.

The cationic conditioning polymer can be included in the compositions at a level by weight of preferably from about 0.01% to about 10%, more preferably from about 0.05% to about 5%.

In the composition of the present invention, the cationic conditioning polymer is preferably water-soluble. By “water soluble” cationic polymer, what is meant is a polymer which is sufficiently soluble in water to form a substantially clear solution to the naked eye at a concentration of 0.05% in water (distilled or equivalent) at 25° C. The preferred polymer will be sufficiently soluble to form a substantially clear solution at 0.1% concentration, more preferably at 0.2% concentration.

The cationic polymers will generally have cationic nitrogen-containing moieties such as quaternary ammonium or cationic amino moieties, and mixtures thereof. The cationic amines can be primary, secondary, or tertiary amines, depending upon the particular species and the pH of the composition. In general, secondary and tertiary amines, especially tertiary amines, are preferred. Amine-substituted vinyl monomers can be polymerized in the amine form, and then optionally can be converted to ammonium by a quaternization reaction. Amines can also be similarly quatemized subsequent to formation of the polymer. For example, tertiary amine functionalities can be quaternized by reaction with a salt of the formula R⁸⁸X wherein R⁸⁸ is a short chain alkyl, preferably a C₁-C₇ alkyl, more preferably a C₁-C₃ alkyl, and X is a salt forming anion as defined above. Any anionic counterions can be utilized for the cationic polymers so long as the water solubility criteria is met. Suitable counterions include halides (e.g., Cl, Br, I, or F, preferably Cl, Br, or I), sulfate, and methylsulfate. Others can also be used, as this list is not exclusive.

Suitable cationic conditioning polymers include, for example: copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16), such as those commercially available from BASF Wyandotte Corp. (Parsippany, N.J., USA) under the LUVIQUAT tradename (e.g., LUVIQUAT FC 370); copolymers of 1-vinyl-2-pyrrolidone and dimethylaminoethyl methacrylate (referred to in the industry by CTFA as Polyquaternium-11) such as those commercially available from Gaf Corporation (Wayne, N.J., USA) under the GAFQUAT tradename (e.g., GAFQUAT 755N); cationic diallyl quaternary ammonium-containing polymers, including, for example, dimethyldiallylammonium chloride homopolymer and copolymers of acrylamide and dimethyldiallylammonium chloride, referred to in the industry (CTFA) as Polyquaternium 6 and Polyquatemium 7, Polyquaternium-7 including that commercially available with a tradename Merquat 550 from Ondeo Nalco; polymethacrylamidopropyl trimonium chloride such as that commercially available with a tradename Polycare 133 from Rhone-Poulenc; and Polyquaternium-37 available from 3V Sigma with tradenames Synthalen CR, Synthalen CU, and Synthalen CN.

Also suitable cationic conditioning polymers herein include cationic cellulose derivatives. Cationic cellulose derivative useful herein include, for example, salts of hydroxyethyl cellulose reacted with trimethyl ammonium substituted epoxide, referred to in the industry (CTFA) as Polyquaternium 10, available from Amerchol Corp. (Edison, N.J., USA) in their Polymer JR® and LR® series, and also available from National Starch & Chemical with a tradename Celquat SC-230M; polymeric quaternary ammonium salts of hydroxyethyl cellulose reacted with lauryl dimethyl ammonium-substituted epoxide, referred to in the industry (CTFA) as Polyquaternium 24, available from Amerchol Corp. (Edison, N.J., USA) under the tradename Polymer LM-200®; and Polyquaternium-4 with tradename Celquat H-100 available from National Starch & Chemical.

Other suitable cationic conditioning polymers include cationic guar gum derivatives, such as guar hydroxypropyltrimonium chloride commercially available from Rhodia in their Jaguar series.

Humectant

The compositions of the present invention can further contain a humectant. The humectants herein are selected from the group consisting of polyhydric alcohols, water soluble alkoxylated nonionic polymers, and mixtures thereof. The humectants herein can be used at levels by weight of the composition of preferably from about 0.1% to about 20%, more preferably from about 0.5% to about 5%.

Polyhydric alcohols useful herein include glycerin, sorbitol, propylene glycol, butylene glycol, hexylene glycol, ethoxylated glucose, 1,2-hexane diol, hexanetriol, dipropylene glycol, erythritol, trehalose, diglycerin, xylitol, maltitol, maltose, glucose, fructose, sodium chondroitin sultate, sodium hyaluronate, sodium adenosin phosphate, sodium lactate, pyrrolidone carbonate, glucosamine, cyclodextrin, and mixtures thereof.

Water soluble alkoxylated nonionic polymers useful herein include polyethylene glycols and polypropylene glycols having a molecular weight of up to about 10,000 such as those with CTFA names PEG-4, PEG-8, PEG-12, PEG-20, PEG-150 and mixtures thereof.

Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: other conditioning agents such as hydrolysed collagen with tradename Peptein 2000 available from Hormel, vitamin E with tradename Emix-d available from Eisai, panthenol available from Roche, panthenyl ethyl ether available from Roche, nonionic surfactants such as glyceryl stearate available from Stepan Chemicals, hydrolysed keratin, proteins, plant extracts, and nutrients; emollients such as PPG-3 myristyl ether with tradename Varonic APM available from Goldschmidt, Trimethyl pentanol hydroxyethyl ether, PPG-11 stearyl ether with tradename Varonic APS available from Goldschmidt, Stearyl heptanoate with tradename Tegosoft SH available from Goldschmidt, Lactil (mixture of Sodium lactate, Sodium PCA, Glycine, Fructose, Urea, Niacinamide, Inositol, Sodium Benzoate, and Lactic acid) available from Goldschmidt, Ethyl hexyl palmitate with tradename Saracos available from Nishin Seiyu and with tradename Tegosoft OP available from Goldschmidt; hair-fixative polymers such as amphoteric fixative polymers, cationic fixative polymers, anionic fixative polymers, nonionic fixative polymers, and silicone grafted copolymers; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; hair oxidizing (bleaching) agents, such as hydrogen peroxide, perborate and persulfate salts; hair reducing agents such as the thioglycolates; perfumes; and sequestering agents, such as disodium ethylenediamine tetra-acetate; ultraviolet and infrared screening and absorbing agents such as octyl salicylate; antidandruff agents such as zinc pyrrithione and salicylic acid; visible particles with tradenames Unisphere and Unicerin available from Induchem AG (Switzerland); and anti-foaming agent such as that with a tradename XS63-B8929 available from GE-Toshiba Silicone.

Product Forms

The hair conditioning compositions of the present invention can be in the form of rinse-off products or leave-on products, can be transparent, translucent, or opaque, and can be formulated in a wide variety of product forms, including but not limited to creams, gels, emulsions, mousses and sprays.

The conditioning compositions of the present invention are especially suitable for rinse-off use. Such compositions are preferably used by following steps:

(i) after shampooing hair, applying to the hair an effective amount of the conditioning composition for conditioning the hair; and

(ii) then rinsing the hair.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Ingredients are identified by chemical or CTFA name, or otherwise defined below.

Compositions (wt %)

| | Ex. 1 | Ex. 2 | Ex. 3 | Ex. 4 | Ex. 5 | Ex. 6 | Ex. 7 |
|---|---|---|---|---|---|---|---|
| Cationic guar gum-1 *1 | 0.7 | 0.7 | 0.7 | 0.5 | 1.0 | 0.7 | — |
| Cationic guar gum-2 *2 | 0.8 | — | — | 0.2 | 0.7 | — | 1.1 |
| Cationic guar gum-3 *3 | — | — | 1.2 | — | — | — | — |
| Nonionic guar gum *4 | — | 0.8 | — | — | — | — | — |
| Hydroxyethylethylcellulose *5 | — | — | 0.8 | 0.8 | — | — | — |
| Cetyl hydroxyethylcellulose *6 | — | — | — | — | — | 0.75 | — |
| Polyquaternium-7 *7 | — | — | — | — | — | — | 0.7 |
| Cetyltrimethylammonium chloride *8 | 1.0 | 1.0 | 1.0 | 1.0 | — | — | — |
| Stearyltrimethylammonium chloride *9 | — | — | — | — | 1.0 | 1.0 | 1.0 |
| Dimethicone copolyol *10 | 2.0 | 2.0 | 2.0 | — | — | 2.0 | 2.0 |
| Amidomethicone copolyol *11 | — | — | — | 2.0 | — | — | — |
| Silicone nanoemulsion *12 | — | — | — | — | 0.6 | — | — |
| PEG-12 *13 | 3.0 | 3.0 | 3.0 | 3.0 | 3.0 | 3.0 | — |
| PEG-150 *14 | — | — | — | — | 0.7 | — | — |
| Anti foaming agent *15 | 0.1 | 0.1 | 0.1 | 0.1 | — | 0.1 | 0.1 |
| Methylchloroisothiazolinone/ | 0.025 | 0.025 | 0.025 | 0.025 | 0.025 | 0.025 | 0.025 |
| Methylisothiazolinone *16 | | | | | | | |
| Methyl Paraben | 0.2 | 0.2 | 0.2 | 0.2 | 0.2 | 0.2 | 0.2 |
| Disodium EDTA | 0.1 | 0.1 | 0.1 | 0.1 | 0.1 | 0.2 | 0.1 |
| Perfume | 0.25 | 0.25 | 0.1 | 0.1 | 0.1 | 0.1 | 0.1 |
| Deionized Water | q.s. to 100% | | | | | | |
| | | | Ex. | Ex. | Ex. | Ex. | Ex. |
| | Ex. 8 | Ex. 9 | 10 | 11 | 12 | 13 | 14 |
| Cationic guar gum-1 *1 | 0.7 | 0.7 | 0.7 | 0.7 | 0.7 | 0.7 | — |
| Nonionic guar gum *4 | 0.9 | 0.9 | 1.0 | 1.0 | 0.9 | 0.9 | 0.9 |
| Polyquaternium-37 *17 | — | — | — | — | — | — | 0.7 |
| Cetyltrimethylammonium chloride *8 | 1.0 | 1.0 | 1.0 | 1.0 | 0.5 | 1.0 | 0.8 |
| Olealkonium chloride *18 | — | — | — | — | 1.0 | — | — |
| Babassuamidopropalkonium | — | — | — | — | — | 1.0 | — |
| chloride *19 | | | | | | | |
| Amidomethicone copolyol *11 | 4.0 | — | 3.0 | 3.0 | 3.0 | 4.0 | 4.0 |
| Amidomethicone copolyol-2 *20 | — | 2.0 | — | — | — | — | — |
| Quaternized silicone nanoemulsion *21 | — | — | 1.0 | — | — | — | — |
| Amodimethicone nanoemulsion *22 | — | — | — | 1.0 | — | — | — |
| Quaternized silicone *23 | — | — | — | — | 2.0 | — | — |
| Anti foaming agent *15 | — | 0.075 | — | — | — | — | — |
| Panthenyl ethyl ether *24 | — | — | — | — | — | 0.05 | — |
| Pantenol *25 | — | — | — | — | — | 0.05 | — |
| Methylchloroisothiazolinone/ | 0.033 | 0.033 | 0.033 | 0.033 | 0.033 | 0.033 | 0.033 |
| Methylisothiazolinone *16 | | | | | | | |
| Methyl Paraben | 0.2 | 0.2 | 0.2 | 0.2 | 0.2 | 0.2 | 0.2 |
| Disodium EDTA | 0.13 | 0.13 | 0.13 | 0.13 | 0.13 | 0.13 | 0.13 |
| Perfume | 0.25 | 0.25 | 0.25 | 0.25 | 0.25 | 0.25 | 0.25 |
| Deionized Water | q.s. to 100% | | | | | | |
| | Ex. | Ex. | Ex. | Ex. | Ex. | Ex. | Ex. |
| | 15 | 16 | 17 | 18 | 19 | 10 | 21 |
| Cationic guar gum-1 *1 | 0.5 | — | — | — | — | — | — |
| Nonionic guar gum *4 | 1.2 | 1.6 | 1.7 | 1.7 | 1.7 | 1.6 | 1.6 |
| Polyquaternium-37 *17 | — | — | — | — | — | — | — |
| Cetyltrimethylammonium chloride *8 | 0.5 | 0.5 | 0.3 | 0.5 | 0.7 | 0.5 | 0.5 |
| Olealkonium chloride *18 | 1.0 | 1.0 | 0.3 | — | 1.0 | 1.0 | 1.0 |
| Babassuamidopropalkonium chloride | — | — | — | 1.0 | — | — | — |
| *19 | | | | | | | |
| Palmitamidopropyltrimonium chloride | — | 0.3 | 0.9 | — | — | — | — |
| *26 | | | | | | | |
| Ceteth-10 *27 | — | — | — | — | 1.0 | 1.0 | — |
| Amidomethicone copolyol *11 | 4.0 | 2.0 | 4.0 | 4.0 | 6.0 | 3.0 | 3.0 |
| Amidomethicone copolyol-2 *20 | — | 2.0 | — | — | — | — | — |
| Quaternized silicone nanoemulsion *21 | — | — | — | — | — | 1.0 | — |
| Amodimethicone nanoemulsion-2 *28 | — | — | — | — | — | — | — |
| Amodimethicone nano emulsion-3 *29 | — | — | — | — | — | — | 1.0 |
| Anti foaming agent *15 | — | 0.075 | — | — | — | — | — |
| Methylchloroisothiazolinone/ | 0.033 | 0.033 | 0.033 | 0.033 | 0.033 | 0.033 | 0.033 |
| Methylisothiazolinone *16 | | | | | | | |
| Methyl Paraben | 0.2 | 0.2 | 0.2 | 0.2 | 0.2 | 0.2 | 0.2 |
| Disodium EDTA | 0.13 | 0.13 | 0.13 | 0.13 | 0.13 | 0.13 | 0.13 |
| Perfume | 0.25 | 0.25 | 0.25 | 0.25 | 0.25 | 0.25 | 0.25 |

| | |
|---|---|
| Deionized Water | q.s. to 100% |

| | Ex. 22 | Ex. 23 |
|---|---|---|
| Cationic guar gum-1 *1 | — | — |
| Nonionic guar gum *4 | 1.6 | 1.4 |
| Polyquaternium-37 *17 | — | 0.3 |
| Cetyltrimethylammonium chloride *8 | 0.5 | 0.5 |
| Olealkonium chloride *18 | 1.0 | 0.8 |
| Babassuamidopropalkonium chloride | — | — |
| *19 | | |
| Palmitamidopropyltrimonium chloride | — | — |
| *26 | | |
| Ceteth-10 *27 | — | 1.0 |
| Amidomethicone copolyol *11 | 2.0 | 4.0 |
| Amidomethicone copolyol-2 *20 | — | — |
| Quaternized silicone nanoemulsion *21 | — | — |
| Amodimethicone nanoemulsion-2 *28 | 2.0 | — |
| Amodimethicone nano emulsion-3 *29 | — | — |
| Anti foaming agent *15 | | |
| Methylchloroisothiazolinone/ | 0.033 | 0.033 |
| Methylisothiazolinone *16 | | |
| Methyl Paraben | 0.2 | 0.2 |
| Disodium EDTA | 0.13 | 0.13 |
| Perfume | 0.25 | 0.25 |

| | |
|---|---|
| Deionized Water | q.s. to 100% |

Definitions of Components
*1 Cationic guar gum-1: Guar hydroxypropyl trimonium chloride having a molecular weight of about 1,500,000 and a level of cationic substitution of 0.15, available from Rhodia with a tradename Jaguar Excel
*2 Cationic guar gum-2: Guar hydroxypropyl trimonium chloride having a molecular weight of about 2,200,000 and a level of cationic substitution of 0.25, available from Rhodia with a tradename Jaguar C-17
*3 Cationic guar gum-3: Guar hydroxypropyl trimonium chloride having a molecular weight of from about 1,000,000 to about 1,500,000 and a level of cationic substitution of about 0.25, available from Rhodia with a tradename Jaguar CHJ
*4 Nonionic guar gum: Jaguar HP-105 having a molecular weight of about 2,000,000 available from Rhodia
*5 Hydroxyethylethylcellulose: Elfacos CD481 available from Akzo Nobel
*6 Cetyl hydroxyethylcellulose: Polysurf 67 available from Aqualon
*7 Polyqyaternium-7: Merquat 550 available from Ondeo Nalco
*8 Cetyl trimethyl ammonium chloride: CTAC 30KC available from KCI
*9 Stearyl trimethyl ammonium chloride: Genamine STACP available from Clariant
*10 Dimethicone copolyol: PEG/PPG-15/15 Dimethicone with a tradename Silicone DC-5330 available from Dow Corning
*11 Amidomethicone copolyol: Silicone BY16-906 available from Dow Corning
*12 Silicone nanoemulsion: Silicone DC-8177 available from Dow Corning
*13 PEG-12: Available from Dow Chemical
*14 PEG-150: Crothix available from Croda
*15 Anti-foaming agent: XS63-B8929 available from GE-Toshiba Silicone
*16 Methylchloroisothiazolinone/ Methylisothiazolinone: Kathon CG available from Rohm&Haas
*17 Polyquaternium-37: Available from 3V Sigma with a tradename Synthalen CR
*18 Olealkonium chloride: Available from Croda with a tradename Incroquat O-50
*19 Babassuamidopropalkonium chloride: Available from Croda with a tradename Incroquat BA-85
*20 Amidomethicone copolyol-2: Available from Dow Corning with a tradename DC2-8500
*21 Quaternized silicone nanoemulsion: Available from Dow Corning with a tradename DC5-7133
*22 Amodimethicone nanoemulsion: Available from General Electric with a tradename XS65-B6413
*23 Quaternized silicone: Available from Noveon wi666th a tradename Ultrasil Q-Plus
*24 Panthenyl ethyl ether: Available from Roche
*25 Panthenol: Available from Roche
*26 Palmitamidopropyltrimonium chloride: Varisoft PATC available from Degussa
*27 Ceteth-10: Available from Nikko
*28 Amodimethicone nanoemulsion-2: DC2-8168 available from Dow Corning
*29 Amodimethicone nano emulsion-3: DC2-8194 available from Dow Corning

Method of Preparation

The hair conditioning compositions of “Ex.1” to “Ex.23” as shown above can be prepared by any conventional method well known in the art. They are suitably made as follows:

The polymeric materials are dispersed in water at room temperature, mixed with vigorous agitation, and heated to 50-70° C. Cationic surfactants, and if included, nonionic surfactants, humectants, and other temperature insensitive components are added to the mixture with agitation. Then the mixture is cooled down to below 40° C., and then the remaining components such as silicones, perfumes, preservatives, and anti-foaming agents, if included, are added to the mixture with agitation.

Examples 1 through 23 are hair conditioning compositions of the present invention which are particularly useful for rinse-off use. These examples have many advantages. For example, the compositions of “Ex.1” through “Ex.23” have a suitable rheology for conditioning compositions, and provide conditioning benefits, especially softness and reduced tangling on wet hair. The compositions of “Ex.1” through “Ex.23” have transparent or translucent appearance. The composition of “Ex.1” through “Ex.23” can provide the above rheological and conditioning benefits while not weighing down the hair. When used in a form of rinse-off products, the compositions of “Ex.1” through “Ex.23” can provide easy to rinse-off feel while providing the above rheological and conditioning benefits.

What is claimed is:

1. A hair conditioning composition comprising by weight:
(a) from about 0.01% to about 10% of a thickening polymer system selected from the group consisting of a cationic thickening polymer, a nonionic thickening polymer, and mixtures thereof;
(b) from about 0.05% to about 10% of a cationic surfactant system selected from the group consisting of one cationic surfactant, a mixture of two or more cationic surfactants, and a mixture of a cationic surfactant and a nonionic surfactant; and
(c) an aqueous carrier;
wherein the composition is substantially free of a water-insoluble high melting point oily compound and an anionic compound.

2. The hair conditioning composition of claim 1 wherein the composition is transparent or translucent.

3. The hair conditioning composition of claim 1 wherein the composition is transparent.

4. The hair conditioning composition of claim 1 wherein the cationic thickening polymer is a cationic guar polymer, and the nonionic thickening polymer is a nonionic guar polymer.

5. The hair conditioning composition of claim 4 wherein the cationic guar polymer has a molecular weight of from about 100,000 to about 3,000,000, and the nonionic guar polymer has a molecular weight of from about 1,000,000 to about 4,000,000.

6. The hair conditioning composition of claim 4 wherein the thickening polymer comprises: from about 0.1% to about 4%, by weight of the composition, of the cationic guar polymer; and from about 0.1% to about 3%, by weight of the composition, of the nonionic guar polymer.

7. The hair conditioning composition of claim 1 comprising from about 0.6% to about 3% of the thickening system.

8. The hair conditioning composition of claim 1 wherein the cationic surfactant is a mono-alkyl trimethyl ammonium salt.

9. The hair conditioning composition of claim 8 wherein the cationic surfactant has a mono-alkyl chain having from 16 to 22 carbon atoms.

10. The hair conditioning composition of claim 1 further comprising from about 0.1% to about 20% of a silicone compound.

11. The hair conditioning composition of claim 10 wherein the silicone compound is selected from the group consisting of a water-soluble silicone compound, a silicone nanoemulsion with an average particle diameter of less than 220 nm, and mixtures thereof.

12. The hair conditioning composition of claim 11 wherein the silicone compound is a water-soluble silicone compound selected from the group consisting of PEG-12 Methyl Ether/Lauroxy PEG-5 Amidopropyl Dimethicone, Bis (C13-15 Alkoxy) PG Amodimethicone, and mixtures thereof.

13. The hair conditioning composition of claim 1 further comprising from about 0.1% to about 20% of a humectant.

14. The hair conditioning composition of claim 1 further comprising from about 0.01% to about 10% of a cationic hair conditioning polymer.

15. The hair conditioning composition of claim 1 having a viscosity of from about 2,000 to about 50,000 mPa·s.

16. The hair conditioning composition of claim 1 which is for rinse-off use.

17. A method of conditioning hair, the method comprising following steps:
(i) after shampooing hair, applying to the hair an effective amount of the hair conditioning composition of claim 1; and
(ii) then rinsing the hair.

18. A hair conditioning composition comprising by weight:
(a) from about 0.5% to about 5% of a thickening polymer system selected from the group consisting of a cationic thickening polymer, a nonionic thickening polymer, and mixtures thereof;
(b) from about 0.25% to about 7% of a cationic surfactant system selected from the group consisting of one cationic surfactant, a mixture of two or more cationic surfactants, and a mixture of a cationic surfactant and a nonionic surfactant;
(c) from about 0.1% to about 10% of a silicone compound selected from the group consisting of a water-soluble silicone compound, a silicone nanoemulsion with an average particle diameter of less than 220 nm, and mixtures thereof; and
(d) an aqueous carrier;
wherein the composition is substantially free of a water-insoluble high melting point oily compound and an anionic compound.

19. A hair conditioning composition comprising by weight:
(a) from about 0.5% to about 5% of a nonionic thickening polymer;
(b) from about 0.25% to about 7% of a cationic surfactant system selected from the group consisting of one cationic surfactant, a mixture of two or more cationic surfactant, and mixture of a cationic surfactant and a nonionic surfactant;
(c) from about 0.1% to about 10% of a silicone compound selected from the group consisting of a water-soluble silicone compound, a silicone nanoemulsion with an average particle diameter of less than 220 nm, and mixtures thereof; and
(d) an aqueous carrier;
wherein the composition is substantially free of a water-insoluble high melting point oily compound, an anionic compound, and a cationic thickening polymer.

20. The hair conditioning composition of claim 19 wherein the nonionic thickening polymer is a nonionic thickening guar polymer.
